(19) United States
(12) Patent Application Publication
(10) Pub. No.: US 2012/0134933 A1
(43) Pub. Date: May 31, 2012

(54) USE OF HIGH-DOSES OF MONOMERIC CONTRAST MEDIUM CONTAINING IODINE IN X-RAY DIAGNOSTICS, IN PARTICULAR IN INTERVENTIONAL X-RAY DIAGNOSTICS AND IN RADIATION THERAPY ASSISTED BY CONTRAST MEDIA CONTAINING IODINE

(75) Inventors: Gregor Jost, Berlin (DE); Hubertus Pietsch, Kleinmachnow (DE); Martin Sieber, Berlin (DE); Matthias Bräutigam, Berlin (DE); Sven Golfier, Berlin (DE)

(73) Assignee: BAYER PHARMA AKTIENGESELLSCHAFT, Berlin (DE)

(21) Appl. No.: 13/319,373

(22) PCT Filed: May 6, 2010
(86) PCT No.: PCT/EP2010/002769

§ 371 Date: Feb. 8, 2012

(30) Foreign Application Priority Data
May 12, 2009 (DE) 10 2009 021 752.5

(51) Int. Cl. A61K 49/04; A61K 31/167; A61P 13/12; C07C 237/46

(52) U.S. Cl. 424/9.454; 564/153; 514/616

(57) ABSTRACT

The invention relates to a diagnostic or therapeutic composition comprising a monomeric X-ray contrast medium containing iodine, in particular iopromide, for use in an X-ray assisted diagnosis or therapy and for the use of high doses of an X-ray contrast medium given to a patient, in particular patients with restricted kidney function.

Modern iodine-containing X-ray contrast media (XCMs) are based on triiodinated aromatic compounds and are comparable in their basic molecular structure. Nowadays, use is made of either monomeric XCMs (a triiodinated aromatic compound) or dimeric XCMs (two linked triiodinated aromatic compounds) as contrast media. (1) At the same iodine concentration, monomeric XCMs have a lower viscosity, but a higher osmolality, compared to dimeric XCMs. Monomeric XCMs differ only slightly in their viscosity and osmolality. For instance, iopromide has only a slightly lower viscosity and osmolality compared to other monomers, such as iohexyl or iopamidol for example. (2)

After administration of XCMs, they are excreted very rapidly via the kidney and, although they are generally tolerated very well (3,4), can result in damage to the kidney by a mechanism which is not fully understood. XCM-induced nephropathy is the third most common cause of acute renal failure. (5) Patients already suffering from kidney failure, i.e., for example patients with diabetes mellitus, have a considerably increased risk of developing XCM-induced nephropathy. (5) Despite extensive research, the pathogenesis of XCM-induced nephropathy is still largely unknown. (6) It is likely that XCM-induced nephropathy is a multifactorial event. One of the causes discussed is a change in renal perfusion associated with induction of regional hypoxia, caused by XCM administration. (7-9) Also discussed as a possible pathogenic mechanism is the macula densa mechanism or tubuloglomerular feedback (TGF), influenced by the osmolality of XCMs. (9) In addition, direct cytotoxic effects, such as reduced cell activity and induction of apoptosis in tubular cells, have also been described in the literature. (10-12) Since the possible pathogenic mechanisms, such as hypoxia or cytotoxic effects of the XCMs, act in a time- and concentration-dependent manner, the local concentration in the kidney and, more particularly, the duration of exposure of the kidney to XCMs is an important factor for renal safety. In some clinical studies, prolonged retention of XCMs is even considered to be prognostic of XCM-induced renal damage. (13, 14)

In vitro studies support the significance of XCM concentration and duration of XCM exposure in the pathogenesis of XCM-induced nephropathy. It was shown in the in vitro investigations that a possible toxic effect is dependent on the duration and concentration of the XCM used. This has been investigated on the basis of multiple studies and end points. (11, 12) For example, Heinrich et al. showed a dose- and time-dependent inactivation of mitochondrial activity. (12) The higher the concentration and the longer the exposure to the XCM, the greater the inhibition of mitochondrial activity. No significant differences were observed between the nonionic XCMs. Similar results were also observed with other end points, such as, for example, the induction of apoptosis or the adenosine triphosphate (ATP) level. (11) Thus, it can be established that a shorter retention time or exposure is indicative of less renal damage. It has been learnt from in vitro and in vivo experiments that differences in the tolerance of modern XCMs depend substantially on rapid excretion.

It is known that monomeric XCMs remain in the kidney for a shorter period than dimeric XCMs. This was demonstrated in case studies in patients and in animal experiments. (13-20) This was dependent on the dose and the renal status of the animals. Thus, the largest differences between the monomeric XCMs and the dimeric XCMs were observed at a high dose and, likewise, in animals with kidney failure. (20) For example, the differences in contrast medium retention for monomeric XCMs and dimeric XCMs in ZSF1 rats with kidney failure were significantly greater than in animals with healthy kidneys. The respective retention of the monomeric XCM and dimeric XCM in ZSF1 rats with kidney failure was also in each case significantly longer than in animals with healthy kidneys. (20) The formulation administered was not a factor.

In the animal studies, prolonged retention time, i.e. higher exposure, correlated with increased damage to the kidney, as predicted in a cell culture experiment. This is indicated by the expression of two biomarkers for renal damage (kidney injury molecule 1 (KIM1) and hemoxygenase 1 (HO1)). Kim1 is strongly expressed in tubular damage, and HO1 is specific for hypoxia in the kidney. In both cases, increased expression is thus indicative of renal damage. (20)

It is unknown to date that there are also distinct differences in the degree of retention in the kidney within the group comprising monomeric XCMs. Parameters and properties, known to date, of these compounds showed no distinct differences. However, our experiments showed differences in the retention in the kidney and in the degree of severity of the morphological changes induced. The clinical surrogate marker for renal damage used to date, serum creatinine, has been found to be too inaccurate to quantify the degree of XCM-induced renal damage. (21) Since, in patients without administration of contrast medium, a similarly large rise in serum creatinine is also observed, as in patients who have developed a supposedly XCM-induced nephropathy after administration of contrast medium.

It was found that, surprisingly, iopromide (Ultravist) at high and very high dosages, as is used or can be used in interventional diagnostics, more particularly interventional coronary angiography, and in XCM-enhanced radiation therapy, is excreted the fastest from the kidney compared to dimeric XCMs and also to other monomeric XCMs. The shortest retention time associated therewith results, surprisingly, in a distinctly lower exposure to the kidney. Compared to all other XCMs, no or distinctly fewer morphological changes were found in the kidney (vacuoles) after iopromide (Ultravist) administration. Since the physicochemical and structural properties of the XCMs, more particularly the nonionic monomeric iodine-containing XCMs, are comparable, the effect described here is surprising and therefore not foreseeable. It has to be assumed that the differences between the monomeric contrast media in animals with kidney failure are even more marked. As has been observed for the difference between monomeric and dimeric XCMs in an animal model.

High-dose use of iodine-containing X-ray contrast media in X-ray diagnostics or XCM-supported radiation therapy is understood to mean dosages of 0.6-2 g of iodine per kg or 1-7 g of iodine per kg at very high dosages.

In particular, the iodine-containing XCM iopromide (Ultravist) has, compared to other monomeric XCMs, advantages with regard to renal tolerance in the following applications:
1. when using multiple or repeated administration to confirm a diagnosis in acute pathological disease states, more particularly in patients with kidney failure,
2. when using multiple or repeated administration to carry out one or more interventions in acute pathological disease states, more particularly in patients with kidney failure,
3. multiple or repeated administration as used in XCM-supported radiation therapy, more particularly in patients with kidney failure,
4. at high dosages of 0.6-2 g of iodine per kg of body weight, or very high dosages of 1-7 g of iodine per kg of body weight, to achieve sufficient quality of diagnosis and a therapeutic effect, as are required in interventional X-ray diagnostics, more particularly in patients with kidney failure.

Iopromide is known to a person skilled in the art, is marketed as Ultravist, and is (1) N,N′-bis(2,3-dihydroxypropyl)-2,4,6-triiodo-5-[(methoxyacetyl)amino]-N-methyl-1,3-benzenedicarboxamide;

### (2) N,N′-bis(2,3-dihydroxypropyl)-2,4,6-triiodo-5-(2-methoxyacetamido)-N-methylisophthalamide

The invention relates to a diagnostic or therapeutic composition comprising a monomeric X-ray contrast medium for the X-ray-supported diagnosis or treatment of patients with limited renal function or kidney failure and/or for the prevention of nephropathies.

The invention also relates to a diagnostic or therapeutic composition as described above, wherein the X-ray contrast medium is selected from the group consisting of iopromide, iohexyl, and iopamidol.

Furthermore, the invention relates to a diagnostic or therapeutic composition as described above, wherein the X-ray contrast medium is iopromide.

The invention also relates to the compositions as described above for high-dose use of monomeric contrast media, more particularly iopromide, for diagnosis or XCM-supported radiation therapy.

Also included is a method for X-ray supported diagnosis or treatment, wherein one of the above-described compositions is used.

The invention further relates to a method as described above, wherein iopromide is used as an X-ray contrast medium and a dose from 0.6 g of iodine per kg to 2 g of iodine per kg is used.

The invention likewise relates to a method as described above, wherein iopromide is used as an X-ray contrast medium at a dose from 1 g of iodine per kg to 7 g of iodine per kg.

The invention also relates to the methods as described above for high-dose use of monomeric contrast media, more particularly iopromide, for diagnosis or XCM-supported radiation therapy.

Also included is a method for preparing a diagnostic or therapeutic composition for X-ray-supported diagnosis or treatment for patients with limited renal function or kidney failure, using a monomeric X-ray contrast medium.

Patients with limited renal function or nephropathies may exhibit, inter alia, the following underlying diseases: hypertension, heart failure, cardiogenic shock, anemia, diabetes mellitus, multiple myeloma.

Accordingly, the invention also relates to the compositions and methods described herein for use in patients with hypertension, heart failure, cardiogenic shock, anemia, diabetes mellitus and/or multiple myeloma.

The invention also relates to a method for preparing a diagnostic or therapeutic composition as described above, wherein the X-ray contrast medium is selected from the group consisting of iopromide, iohexyl, iomeprol, and iopamidol.

The invention also relates to the methods for preparing a diagnostic or therapeutic composition as described above for high-dose use of monomeric contrast media, more particularly iopromide, for diagnosis or XCM-supported radiation therapy.

The invention relates to all diagnostic compositions as described in this text, preferably used as contrast media for X-ray diagnostics, having the following composition:
base substance having a contrast-conferring element, such as iodine, preferably monomeric contrast media, more particularly iopromide, iohexyl, iomeprol, or iopamidol, particularly preferably iopromide,
buffer, such as trometamol,
chelating agent, such as EDTA, DTPA,
water for injection,
salts of magnesium, potassium, calcium, or sodium.

The invention relates particularly to the preparation according to the invention or to the use according to the invention of all formulations of Ultravist, such as, for example:

Composition of Ultravist 150

Per 1 ml of solution:

| | | |
|---|---|---|
| 311.70 | mg | iopromide |
| 0.10 | mg | sodium calcium edetate |
| 2.42 | mg | trometamol |
| 5.60 | mg | hydrochloric acid, 10% |
| 843.18 | mg | water for injection |

Composition of Ultravist 240

Per 1 ml of solution:

| | | |
|---|---|---|
| 498.72 | mg | iopromide |
| 0.10 | mg | sodium calcium edetate |
| 2.42 | mg | trometamol |
| 5.60 | mg | hydrochloric acid, 10% |
| 755.46 | mg | water for injection |

Composition of Ultravist 300

Per 1 ml of solution:

| | | |
|---|---|---|
| 623.40 | mg | iopromide |
| 0.10 | mg | sodium calcium edetate |
| 2.42 | mg | trometamol |
| 5.60 | mg | hydrochloric acid, 10% |
| 696.78 | mg | water for injection |

Composition of Ultravist 370

Per 1 ml of solution:

| | | |
|---|---|---|
| 768.86 | mg | iopromide |
| 0.10 | mg | sodium calcium edetate |
| 2.42 | mg | trometamol |
| 5.60 | mg | hydrochloric acid, 10% |
| 628.72 | mg | water for injection |

Patients with limited renal function or renal failure

Renal failure is a deterioration or loss of renal function. The leading symptom is a reduction in urea secretion with oliguria/anuria and a rise in the retention values for urea and creatinine.

Depending on the time course, there are two forms of renal failure:
chronic renal failure
acute renal failure

In both cases, the kidneys no longer function qualitatively or function only to a limited extent. Acute renal failure can arise over the course of acute deterioration of an already existing renal disease, such as diabetic or hypertensive renal damage, or as a result of chronic glomerulonephritis. Acute renal failure may also occur as a result of acute glomerulonephritis, autoimmune disease, infections or following toxic renal damage, etc. Important triggers of toxic renal failure are not only myolysis, hemolysis, various cytostatics, but also X-ray contrast media.

Acute renal failure is a severe disease and requires intensive care. After treatment of the underlying disease, the therapeutic priority is the stabilization of the circulation and electrolyte balance. In particular, however, the administration of all medicaments which are potentially damaging to the kidney (including contrast media) must be minimized or avoided.

Chronic renal failure can, during progression into the terminal stage, ultimately guide the permanent cessation of renal function. The most common causes are chronic glomerulonephritis, type 2 diabetes mellitus/diabetic nephropathy, high blood pressure, inflammations, and renal infections.

Chronic renal failure develops over the course of months to years. Symptoms generally appear only at a very advanced stage. In the event of permanent loss of renal function, the treatment carried out is dialysis or kidney transplantation.

Depending on the duration of the disorder present, a distinction is made between acute renal failure and chronic renal failure.

A distinction is made between two independent criteria for chronic renal failure:
renal damage for ≧3 months, defined by structural or functional disorders of the kidney, with or without reduced GFR
GFR <60 ml/min/1.73 m2 for ≧3 months, with or without renal damage

GFR is the best overall index of normal or pathological renal function. However, the GFR varies depending on age, sex, size, and body.

Stages:

Depending on the renal function and of a thus preexisting condition, the following divisions/classifications can be made:

| | | GFR ml/min/1.73 m2 |
|---|---|---|
| Stage | Description | (renal function) |
| 1 | Renal damage with normal or elevated | GFR ≧90 |
| 2 | Slight kidney failure | GFR 60-89 |
| 3 | Moderate kidney failure | GFR 30-59 |
| 4 | Severe kidney failure | GFR 15-29 |
| 5 | Renal failure | <15 (or dialysis) |

Contrast medium-induced renal failure is defined as follows:

Deterioration of renal function occurring within 3 days after CM administration and to the exclusion of other etiological circumstances, characterized by a rise in serum creatinine of more than 25% or 0.5 mg/dl relative to the starting value.

The following preexisting conditions or interactions with medicaments form the risk profile for the development of nephropathies:
hypertension
heart failure, caridiogenic shock
age
anemia
diabetes mellitus
contrast medium volume
multiple myeloma

The following clinical values indicate limited renal function: serum creatinine >1.5 mg/dl or GFR <60 ml/min/1.73 m2.

In principal, the volume administered is considered to be a possible risk factor for iodinated X-ray contrast media. Accordingly, the volume should be minimized for high-risk patients. Higher contrast medium volumes (>100 ml) are associated with a higher rate of secondary effects, particularly in high-risk patients. But also small (about 30 ml) volumes can, in patients at high risk, lead to acute renal failure being induced and dialysis being required.

Patients with limited renal function (class 3 to 5) are considered to be high-risk patients for administration of an X-ray contrast medium.

### DESCRIPTION OF THE FIGURES

FIG. 1: Renal iodine content 24 hours after injection of XCM. 6 Wistar Han rats were each injected with iopromide 300, iomeprol 300, and iohexyl 350 (4 g of iodine per kg of body weight (BW)). 24 hours after the injection, the respective iodine content was determined by means of X-ray fluorescence analysis (XFA). The significantly lowest iodine contents were observed after administration of iopromide 300. The control used was physiological saline. (*p<0.005)

FIG. 2: Renal cortex iodine content 24 hours after injection of XCM. 6 Wistar Han rats were each injected with iopromide 300, iomeprol 300, and iohexyl 350 (4 g of iodine per kg of BW). 24 hours after the injection, the respective iodine content was determined in the renal cortex by means of X-ray computed tomography (CT). The significantly lowest values were observed after administration of iopromide 300. The control used was physiological saline. (*p<0.005)

FIG. 3: Vacuolization in the kidney 24 hours after injection of XCM. Hematoxylin and eosin (HE) staining of a paraffin section of the kidney 24 hours after administration of contrast medium. 6 Wistar Han rats were each injected with iopromide 300, iomeprol 300, and iohexyl 350 (4 g of iodine per kg of BW). The right-hand column is an enlargement of the left-hand column. After administration of iohexyl, but also of iomeprol, increased vacuolization can be observed.

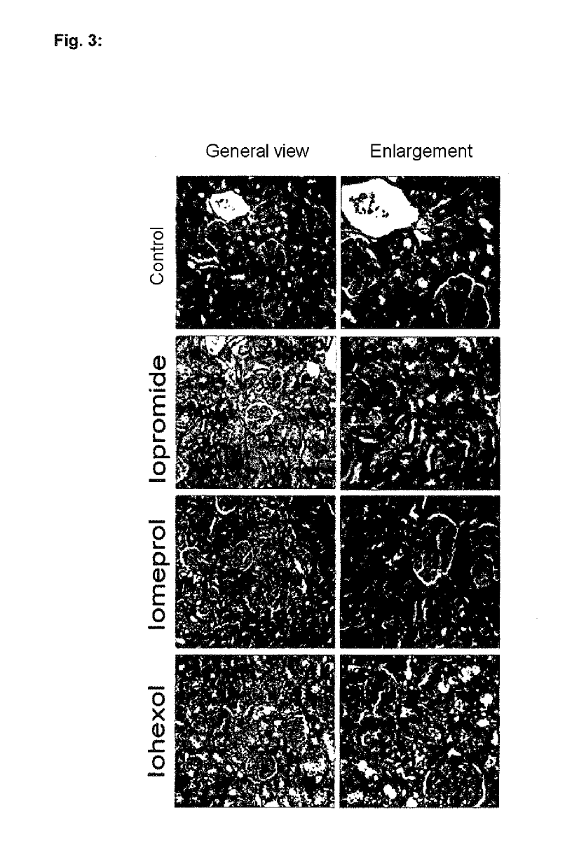

### EXAMPLES

### Lower Exposure of the Kidney after Iopromide Treatment Compared to Treatment with Other Monomeric XCMs

Materials and Methods:

Contrast Media:

For the study, use was made of the following XCMs, each obtained from their producer. The monomeric XCMs investigated were iopromide 300 (Bayer Vital, Leverkusen), iomeprol 300 (Altana, Konstanz, Germany), and iohexyl 350 (Omnipaque, GE Healthcare, Munich, Germany). For comparison, Isovist 300 (Bayer Vital, Leverkusen) and iodixanol 320 and iodixanol 270 (Visipaque, GE Healthcare, Munich, Germany), two dimeric XCMs, were also included in the study. The XCMs were injected in one dose of 4 g per kg of body weight (BW). As with all toxicological issues, it should be noted that the conversion to the human dose has to be based on body surface area. Thus, 4 g of iodine per kg of BW in the rat correspond to about 0.6 g of iodine per kg of BW in humans.

Animal Model:

Wistar Han (CRL: WI) rats (male; 230-300 g) were obtained from Charles River (Sulzfeld, Germany). The animals were kept under normal laboratory conditions at a temperature of 22° C. and a night/day rhythm of 12 hours. The animals had access to standard feed and water ad libitum. The animals were kept and treated in accordance with the German animal welfare guidelines.

Experimental Design:

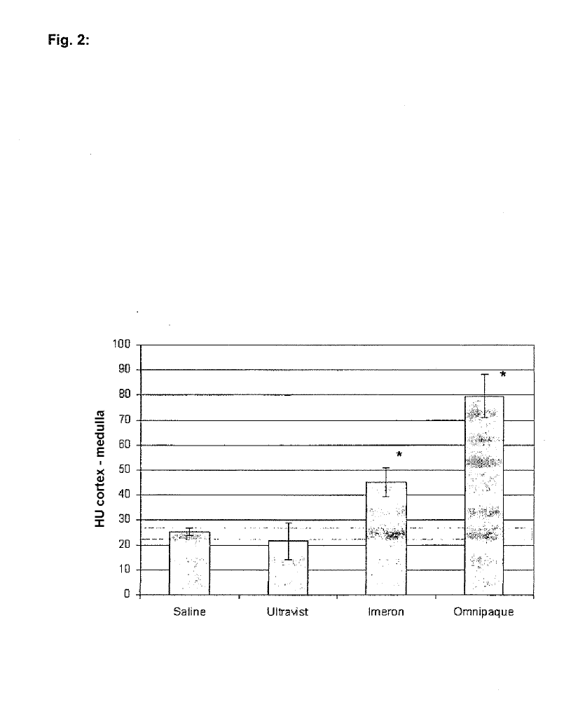

The respective XCMs were intravenously (i.v.) injected in one dose of 4 g of iodine per kg of BW (corresponds to 0.6 g of iodine per kg of BW in humans) into the tail vein. The XCMs were manually injected as a bolus, followed by a 0.2 ml bolus of saline solution. Each test group consisted of 6 test animals. The iodine concentrations in the kidneys were determined ex vivo by X-ray fluorescence analysis (XFA). 24 hours after the XCM injection, the animals were sacrificed and both kidneys were removed. The kidneys were lysed in 10% KOH, and the iodine concentration in the sample was subsequently determined by XFA. The iodine concentrations in the renal cortex were determined 24 hours after the injection using a 64-slice CT scanner (Sensation 64, Siemens Medical Solutions, Erlangen, Germany). Scanner settings (80 kV, 120 mAseff) were used for all investigations, and the reconstructions were carried out with an image field of 70×70 mm and a thickness of 1 mm The X-ray attenuation in Hounsfield units (HU) was determined in the cortex of the kidney in 3 independent regions of interest (ROI) (FIG. 2). All data were carried out by two independent blinded readers.

To determine the degree of vacuolization, a kidney was removed 24 after the injection of the XCMs and a medial piece of tissue was fixed in formaldehyde and embedded in paraffin. The microtome sections were stained with hematoxylin, and the degree of vacuolization was determined. The determination of the degree of vacuolization was carried out in a blinded experiment by Dr. Haider, Institut für Tierpathologie [Institute for Animal Pathology].

Statistics:

Descriptive statistics (mean value, standard deviation, Student's t-test) were calculated using the program Excel (Microsoft, Redmond, Wash., USA).

Results:

Retention of Monomers in the Kidney:

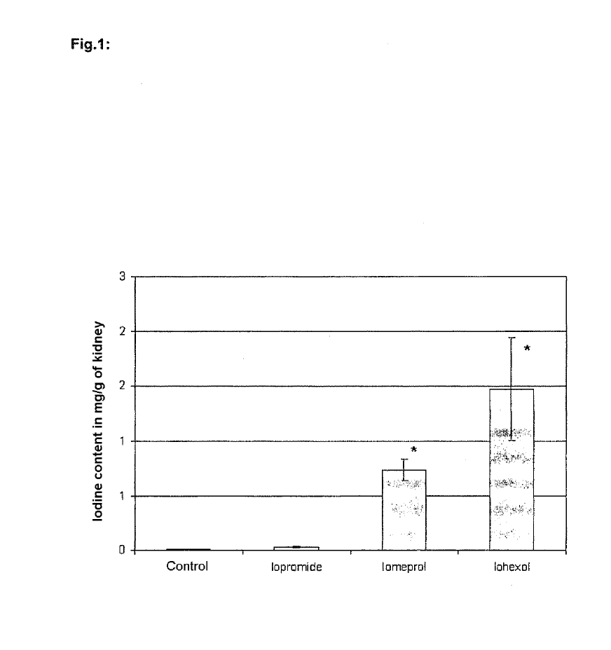

Compared to the treatment with other monomeric XCMs, the significantly lowest iodine concentration (p<0.0005) was found, by means of XFA, after administration of iopromide 300 (0.034+/−0.007 mg of iodine per g of kidney tissue). Higher iodine contents in the kidney were observed after administration of the monomers iomeprol 300 (0.738+/−0.098 mg of iodine per g of kidney tissue) and iohexyl 350 (1.471+/−0.470 mg of iodine per g of kidney tissue). The iodine values for the two dimeric XCMs iotrolan 300 (3.4+/−0.6 mg of iodine per g of kidney tissue) and iodixanol 320 (6.8+/−1.1 mg of iodine per g of kidney tissue) were markedly increased. After administration of sodium chloride, only slight traces of iodine were found (0.007+/−0.004 mg of iodine per g of kidney tissue) (FIG. 1).

Retention of Contrast Media in the Renal Cortex:

At the time point 24 after the injection, the lowest X-ray attenuation in the renal cortex and thus lowest iodine content was found, by means of CT, after administration of iopromide 300 (21.6+/−7.3 HU). These values were in the region of the sodium chloride control (25.2+/−1.7 HU). After administration of the monomers iomeprol 300 and iohexyl 350 were 45.0+/−5.9 HU and 79.5+/−8.6 HU, these values were distinctly above the control. Both values are significantly elevated compared to iopromide treatment (p<0.0005). As already found for the iodine content, the values for the dimeric XCMs were elevated most. The iodine values for the two dimeric XCMs iotrolan 300 (217.2+/−29.4 HU) and iodixanol 320 (359.3 +/−56.8 HU) were markedly increased (FIG. 2).

Vacuolization in Tubular Cells after Administration of Xcms

Vacuolization occurs not only after the administration of XCMs, but also after administration of other drugs. In this process, increased vesicles are formed in the tubular cells. The exact role of this reversible process is largely unknown. However, it is considered to be a sign of delayed excretion. Prolonged retention at higher concentrations leads to increased vacuolization.

Compared to the treatment with other monomeric XCMs, the lowest degree of vacuolization was observed after the administration of iopromide. Two of the animals exhibited no vacuolization and four exhibited slight vacuolization. By contrast, 24 hours after injection with iomeprol 300, slight vacuolization was found in all the animals investigated. And after 24 hours following injection with iohexyl 350, slight vacuolization was observed in 3 animals and moderate vacuolization was observed in 3 animals (table 1).

TABLE 1
Vacuolization 24 after injection of XCM
or of saline as a negative control

| | Sodium chloride | Iopromide | Iomeprol | Iohexol |
|---|---|---|---|---|
| | control | 300 | 300 | 350 |
| No | 3 | 2 | — | — |
| vacuolization | | | | |
| Slight degree | 3 | 4 | 6 | 3 |
| of vacuolization | | | | |
| Moderate degree | — | — | — | 3 |
| of vacuolization | | | | |

### LITERATURE

1. Gries H. X-Ray contrast Agents: physico-chemical properties. Dawson P, Cosgrove D, Graininger R, editors. Oxford: Isis Medical Media Ltd; 1999. 15-22 p.
2. Speck U. X-Ray contrast Agents: physico-chemical properties. Dawson P, Cosgrove D, Graininger R, editors. Oxford: Isis Medical Media Ltd; 1999. 35-46 p.
3. Morcos S K, Thomsen H S, Webb J A. Contrast-media-induced nephrotoxicity: a consensus report. Contrast Media Safety Committee, European Society of Urogenital Radiology (ESUR). Eur Radiol 1999; 9(8): 1602-1613.
4. Thomsen H S. Reducing the risk of contrast media induced nephrotoxicity. Thomsen H S, editor. Heidelberg: Springer; 2006.35-46 p.
5. Barrett B J, Parfrey P S. Clinical practice. Preventing nephropathy induced by contrast medium. N Engl J Med 2006; 354(4): 379-386.
6. Persson P B, Tepel M. Contrast medium-induced nephropathy: the pathophysiology. Kidney Int Suppl 2006(100): S8-10.
7. Heyman S N, Rosenberger C, Rosen S. Regional alterations in renal haemodynamics and oxygenation: a role in contrast medium-induced nephropathy. Nephrol Dial Transplant 2005; 20 Suppl 1: i6-11.
8. Liss P, Nygren A, Erikson U, Ulfendahl H R. Injection of low and iso-osmolar contrast medium decreases oxygen tension in the renal medulla. Kidney Int 1998; 53(3): 698-702.
9. Seeliger E, Flemming B, Wronski T, et al. Viscosity of contrast media perturbs renal hemodynamics. J Am Soc Nephrol 2007; 18(11): 2912-2920.
10. Hizoh I, Strater J, Schick C S, Kubler W, Haller C. Radiocontrast-induced DNA fragmentation of renal tubular cells in vitro: role of hypertonicity. Nephrol Dial Transplant 1998; 13(4): 911-918.
11. Hardiek K, Katholi R E, Ramkumar V, Deitrick C. Proximal tubule cell response to radiographic contrast media. Am J Physiol Renal Physiol 2001; 280(1): F61-70.
12. Heinrich M C, Kuhlmann M K, Grgic A, Heckmann M, Kramann B, Uder M. Cytotoxic effects of ionic high-osmolar, nonionic monomeric, and nonionic iso-osmolar dimeric iodinated contrast media on renal tubular cells in vitro. Radiology 2005; 235(3): 843-849.
13. Love L, Olson M C. Persistent CT nephrogram: significance in the diagnosis of contrast nephropathy—an update. Urol Radiol 1991; 12(4): 206-208.
14. Yamazaki H, Oi H, Matsushita M, et al. Renal cortical retention on delayed CT after angiography and contrast associated nephropathy. Br J Radiol 1997; 70(837): 897-902.
15. Love L, Johnson M S, Bresler M E, Nelson J E, Olson M C, Flisak M E. The persistent computed tomography nephrogram: its significance in the diagnosis of contrast-associated nephrotoxicity. Br J Radiol 1994; 67(802): 951-957.
16. Love L, Lind J A, Jr., Olson M C. Persistent CT nephrogram: significance in the diagnosis of contrast nephropathy. Radiology 1989; 172(1): 125-129.
17. Yamazaki H, Oi H, Matsushita M, et al. Renal cortical retention of contrast medium after angiography as assessed by delayed CT: a multivariate analysis. Radiat Med 1996; 14(5): 247-250.
18. Yamazaki H, Oi H, Matsushita M, et al. Renal cortical retention on delayed CT and nephropathy following transcatheter arterial chemoembolisation. Br J Radiol 2001; 74(884): 695-700.
19. Yamazaki H, Oi H, Matsushita M, et al. Focal residual contrast media in the kidney 24 hours after angiography. Acta Radiol 1996; 37(3 Pt 1): 348-351.
20. Jost G, Pietsch H, Sommer J, et al. Retention of iodine and expression of biomarkers for renal damage in the kidney after application of iodinated contrast media in rats. Invest Radiol 2009; 44(2): 114-123.
21. Newhouse J H, Kho D, Rao Q A, Starren J. Frequency of serum creatinine changes in the absence of iodinated contrast material: implications for studies of contrast nephrotoxicity. AJR Am J Roentgenol 2008; 191(2): 376-382.

1. A diagnostic or therapeutic composition comprising a monomeric iodine-containing X-ray contrast medium for X-ray-supported diagnosis or treatment for the high-dose use of X-ray contrast medium.

2. (canceled)

3. (canceled)

4. The diagnostic or therapeutic composition as claimed in claim 1, wherein the X-ray contrast medium is selected from the group consisting of iopromide, iohexyl, iomeprol, and iopamidol.

5. The diagnostic or therapeutic composition as claimed in claim 4, wherein the X-ray contrast medium is iopromide.

6. A method for X-ray-supported diagnosis or treatment in the high-dose use of X-ray contrast medium in a patient, comprising administering to the patient the composition of claim 1.

7. The method as claimed in claim 6, wherein the composition comprises iopromide and is administered in a dose from 0.6 g of iodine per kg of body weight to 2 g of iodine per kg of body weight.

8. The method as claimed in claim 6, wherein the composition is administered in a dose from 1 g of iodine per kg of body weight to 7 g of iodine per kg of body weight.

9. (canceled)

10. (canceled)

11. (canceled)

12. The method for preparing a diagnostic or therapeutic composition as claimed in claim 9, wherein the X-ray contrast medium is selected from the group consisting of iopromide, iohexyl, iomeprol, and iopamidol.

13. The method for preparing a diagnostic or therapeutic composition as claimed in claim 12, wherein the X-ray contrast medium is iopromide.

14. A method of X-ray-supported diagnosis or treatment comprising administering to a patient in need thereof a monomeric iodine-containing X-ray contrast medium in an amount of from 0.6 g of iodine per kg of body weight to 7 g of iodine per kg of body weight.

15. The method as claimed in claim 14, wherein the X-ray contrast medium is administered in an amount of from 0.6 g of iodine per kg of body weight to 2 g of iodine per kg of body weight.

16. The method as claimed in claim 14, wherein the X-ray contrast medium is administered in an amount of from 1 g of iodine per kg of body weight to 7 g of iodine per kg of body weight.

17. The method as claimed in claim 15, wherein the patient has limited renal function or kidney failure.

18. The method as claimed in claim 15, wherein the X-ray contrast medium is selected from the group consisting of iopromide, iohexyl, iomeprol, and iopamidol.

19. The method as claimed in claim 15, wherein the X-ray contrast medium is iopromide.

20. The method as claimed in claim 16, wherein the patient has limited renal function or kidney failure.

21. The method as claimed in claim 16, wherein the X-ray contrast medium is selected from the group consisting of iopromide, iohexyl, iomeprol, and iopamidol.

22. The method as claimed in claim 16, wherein the X-ray contrast medium is iopromide.

23. A method of preventing nephropathies in a patient for whom X-ray-supported diagnosis or treatment is indicated comprising administering to the patient a monomeric iodine-containing X-ray contrast medium in an amount of from 0.6 g of iodine per kg of body weight to 7 g of iodine per kg of body weight.

24. The method as claimed in claim 23, wherein the X-ray contrast medium is selected from the group consisting of iopromide, iohexyl, iomeprol, and iopamidol.

25. The method as claimed in claim 23, wherein the X-ray contrast medium is iopromide.
